(19) United States
(12) Patent Application Publication
(10) Pub. No.: US 2025/0194980 A1
(43) Pub. Date: Jun. 19, 2025

(54) PROBE AND METHOD FOR SENSING CELLULAR SIGNALS

(71) Applicant: SAMSUNG ELECTRONICS CO., LTD., Suwon-si (KR)

(72) Inventors: Mikyung KIM, Suwon-si (KR); Kitae PARK, Suwon-si (KR); Carlos Cristiano DE JESUS ALCANTARA, Suwon-si (KR); Jonghan KIM, Suwon-si (KR); Chisung BAE, Suwon-si (KR); Anes JU, Suwon-si (KR)

(73) Assignee: SAMSUNG ELECTRONICS CO., LTD., Suwon-si (KR)

(21) Appl. No.: 18/926,260

(22) Filed: Oct. 24, 2024

(30) Foreign Application Priority Data
Dec. 15, 2023 (KR) 10-2023-0183443

(51) Int. Cl. A61B 5/25

(52) U.S. Cl. CPC A61B 5/25

(57) ABSTRACT

A probe apparatus for sensing a cellular signal includes a base layer, an extracellular electrode disposed on the base layer, a first middle layer that is disposed on the base layer and configured to cover the extracellular electrode, an intracellular electrode that is disposed on the first middle layer, a second middle layer that is disposed on the first middle layer and configured to cover the intracellular electrode, a cover layer disposed on the second middle layer, and a fluid-flow channel provided between the second middle layer and the cover layer.

### CROSS-REFERENCE TO RELATED APPLICATIONS

This application claims the benefit under 35 USC § 119(a) of Korean Patent Application No. 10-2023-0183443, filed on Dec. 15, 2023, in the Korean Intellectual Property Office, the entire disclosure of which is incorporated herein by reference for all purposes.

### BACKGROUND

### 1. Field

The following description relates to a probe for sensing cellular signals and a method of sensing the same.

### 2. Description of Related Art

A cell in vivo, for example, may be sensed with intracellular and extracellular sensing methods. There is a need for technology with compact structure to sense a cell's nerve signaling.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, a probe apparatus for sensing cellular signals includes: a base layer; a first electrode disposed on the base layer and including a first electrode head, a first electrode line extending from the first electrode head, and a first electrode terminal extending from the extracellular electrode line; a first middle layer that is disposed on the base layer and is configured to cover the first electrode line; a second electrode that is disposed on the first middle layer and provided spaced apart from the first electrode, wherein the second electrode includes a second electrode head, a second electrode line formed extending from the second electrode head, and a second electrode terminal extending from the second electrode line; a second middle layer that is disposed on the first middle layer and is configured to cover the second electrode line; a cover layer disposed on the second middle layer; and a fluid-flow channel provided between the second middle layer and the cover layer.

The cover layer may include: a cover layer head, which is a longitudinal member; and a cover layer body that is adjacent to the cover layer head and has a width greater than a corresponding width of the cover layer head.

The cover layer may further include: a cover main hole that is formed penetrating the cover layer head and exposes the first electrode head and the second electrode head each to the outside.

The cover layer may further include: a first terminal hole that is formed penetrating the cover layer body and that exposes the first electrode terminal to the outside.

The cover layer may further include: a second terminal hole that is formed penetrating the cover layer body, that exposes the second electrode terminal to the outside, and that is provided at a location spaced apart from the first terminal hole.

The cover layer may further include: a channel hole that is formed penetrating the cover layer body and that communicates the fluid-flow channel to the outside.

The flow channel may be provided with one end open toward the cover main hole and another end open toward the channel hole.

The base layer may include: a base layer head that is configured to support the first electrode head and overlaps the cover layer head; and a base layer body that is connected to the base layer head and is configured to support the first electrode terminal.

The first middle layer may include: a first middle layer head which has a hole that communicates with the cover main hole, that is configured to support the second electrode head, and that overlaps the cover layer head; and a first middle layer body that is connected to the first middle layer head and is configured to support the second electrode terminal.

The second middle layer may include: a second middle layer head which has a hole that communicates to the cover main hole and that overlaps the cover layer head; and a second middle layer body adjacent to the second middle layer head.

There may be multiple first electrodes and multiple second electrodes.

The first electrodes may have different lengths with respect to each other.

A longitudinal direction of the first electrode line and a longitudinal direction of the second electrode terminal may be provided to intersect with each other.

A width of the flow channel may be formed to be less than a width of a portion of the second middle layer that does not overlap the flow channel.

The apparatus may further include: a pump configured to generate pneumatic pressure; and a pump line connecting the pump with the flow channel.

In another general aspect, a probe apparatus for sensing a cellular signal includes: a base layer; a second electrode disposed on the base layer; a first middle layer that is disposed on the base layer and configured to cover at least a portion of the second electrode; a first electrode disposed on the first middle layer and provided spaced apart from the second electrode; a second middle layer that is disposed on the first middle layer and configured to cover at least a portion of the first electrode; a sacrificial layer disposed on the second middle layer and provided spaced apart from the second electrode and the first electrode, the sacrificial layer including a material that dissolves in a liquid; and a cover layer disposed on the sacrificial layer.

A width of the sacrificial layer may be formed to be less than a width of a portion of the second middle layer that does not overlap the sacrificial layer.

In another general aspect, a cell probe includes: a pointed shaft and a body together including a first sensing set; the pointed shaft including a first opening at a pointed end of the shaft; the first sensing set including a first fluid pathway, a first electrical pathway, and a second electrical pathway; the first fluid pathway including a first tunnel passing longitudinally through the shaft and having one end opening at the first hole; the first electrical pathway including a first electrode pad exposed via the first hole and a first electrical line connected to the first electrode pad; and the second electrical pathway including a second electrode pad exposed via the first hole and a second electrical line connected to the second electrode pad.

The first electrical line may be connected to a first electrical terminal included in the body; the second electrical line may be connected to a second electrical terminal included in the body; and the cell probe may further include a pump connected with the first tunnel to allow fluid flow between the pump and the one end of the first tunnel.

The pointed shaft may include a second opening at the pointed end of the shaft, the pointed shaft and the body together may include a second sensing set, and the second sensing set may include: a second fluid pathway, a third electrical pathway, and a fourth electrical pathway; the second fluid pathway may include a second tunnel passing longitudinally through shaft and having one end opening at the second hole; the third electrical pathway including a third electrode pad exposed via the second hole and a third electrical line connected to the third electrode pad; and the fourth electrical pathway including a fourth electrode pad exposed via the second hole and a fourth electrical line connected to the fourth electrode pad.

Other features and aspects will be apparent from the following detailed description, the drawings, and the claims.

### BRIEF DESCRIPTION OF THE DRAWINGS

FIG. 1 illustrates a perspective view of an example probe apparatus for sensing an in vivo intracellular signal, according to one or more embodiments.

FIG. 2 illustrates a perspective view of an example extracellular electrode disposed on a base layer, according to one or more embodiments.

FIG. 3 illustrates a perspective view of an example first middle layer covering the extracellular electrode, according to one or more embodiments.

FIG. 4 illustrates a perspective view of an example intracellular electrode disposed on the first middle layer, according to one or more embodiments.

FIG. 5 illustrates a perspective view of the example intracellular electrode disposed on the first middle layer, according to one or more embodiments.

FIG. 6 illustrates a perspective view of an example second middle layer covering the intracellular electrode, according to one or more embodiments.

FIG. 7 illustrates a perspective view of an example sacrificial layer disposed on the second middle layer, according to one or more embodiments.

FIG. 8 illustrates a perspective view of the example sacrificial layer disposed on the second middle layer, according to one or more embodiments.

FIG. 9 illustrates a perspective view of an example of a cover layer covering the sacrificial layer, according to one or more embodiments.

FIG. 10 illustrates a perspective view after the sacrificial layer is dissolved and removed from FIG. 9, according to one or more embodiments.

FIG. 11 illustrates a perspective view of the cover layer, according to one or more embodiments.

FIG. 12 illustrates a cross-sectional view taken along the line I-I of FIG. 9, according to one or more embodiments.

FIG. 13 illustrates a cross-sectional view taken along the line A-A of FIG. 9, according to one or more embodiments.

FIG. 14 illustrates a cross-sectional view taken along the line A-A of FIG. 9, in which a flow channel is formed, according to one or more embodiments.

FIG. 15 illustrates a cross-sectional view taken along the line B-B of FIG. 10, according to one or more embodiments.

FIG. 16 illustrates an example of a method of using a probe apparatus for sensing an in vivo intracellular signal, according to one or more embodiments.

FIG. 17 illustrates an example of an operation of fixing a cell through a flow channel, according to one or more embodiments.

FIG. 18 illustrates an example of a target cell in the vicinity of the probe apparatus for sensing an in vivo intracellular signal, according to one or more embodiments.

FIG. 19 shows the location of the target cell in the vicinity of the probe apparatus for sensing an in vivo intracellular signal, according to one or more embodiments.

FIG. 20 illustrates a cross-sectional view of an example probe apparatus for sensing an in vivo intracellular signal, according to one or more embodiments.

FIG. 21 illustrates a cross-sectional view of an example probe apparatus for sensing an in vivo intracellular signal, according to one or more embodiments.

FIG. 22 illustrates a perspective view of an example probe apparatus for sensing an in vivo intracellular signal, according to one or more embodiments.

Throughout the drawings and the detailed description, unless otherwise described or provided, the same or like drawing reference numerals will be understood to refer to the same or like elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences of operations described herein are merely examples, and are not limited to those set forth herein, but may be changed as will be apparent after an understanding of the disclosure of this application, with the exception of operations necessarily occurring in a certain order. Also, descriptions of features that are known after an understanding of the disclosure of this application may be omitted for increased clarity and conciseness.

The features described herein may be embodied in different forms and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application.

The terminology used herein is for describing various examples only and is not to be used to limit the disclosure. The articles “a,” “an,” and “the” are intended to include the plural forms as well, unless the context clearly indicates otherwise. As used herein, the term “and/or” includes any one and any combination of any two or more of the associated listed items. As non-limiting examples, terms “comprise” or “comprises,” “include” or “includes,” and “have” or “has” specify the presence of stated features, numbers, operations, members, elements, and/or combinations thereof, but do not preclude the presence or addition of one or more other features, numbers, operations, members, elements, and/or combinations thereof.

Throughout the specification, when a component or element is described as being “connected to,” “coupled to,” or “joined to” another component or element, it may be directly “connected to,” “coupled to,” or “joined to” the other component or element, or there may reasonably be one or more other components or elements intervening therebetween. When a component or element is described as being “directly connected to,” “directly coupled to,” or “directly joined to” another component or element, there can be no other elements intervening therebetween. Likewise, expressions, for example, “between” and “immediately between” and “adjacent to” and “immediately adjacent to” may also be construed as described in the foregoing.

Although terms such as “first,” “second,” and “third”, or A, B, (a), (b), and the like may be used herein to describe various members, components, regions, layers, or sections, these members, components, regions, layers, or sections are not to be limited by these terms. Each of these terminologies is not used to define an essence, order, or sequence of corresponding members, components, regions, layers, or sections, for example, but used merely to distinguish the corresponding members, components, regions, layers, or sections from other members, components, regions, layers, or sections. Thus, a first member, component, region, layer, or section referred to in the examples described herein may also be referred to as a second member, component, region, layer, or section without departing from the teachings of the examples.

Unless otherwise defined, all terms, including technical and scientific terms, used herein have the same meaning as commonly understood by one of ordinary skill in the art to which this disclosure pertains and based on an understanding of the disclosure of the present application. Terms, such as those defined in commonly used dictionaries, are to be interpreted as having a meaning that is consistent with their meaning in the context of the relevant art and the disclosure of the present application and are not to be interpreted in an idealized or overly formal sense unless expressly so defined herein. The use of the term “may” herein with respect to an example or embodiment, e.g., as to what an example or embodiment may include or implement, means that at least one example or embodiment exists where such a feature is included or implemented, while all examples are not limited thereto.

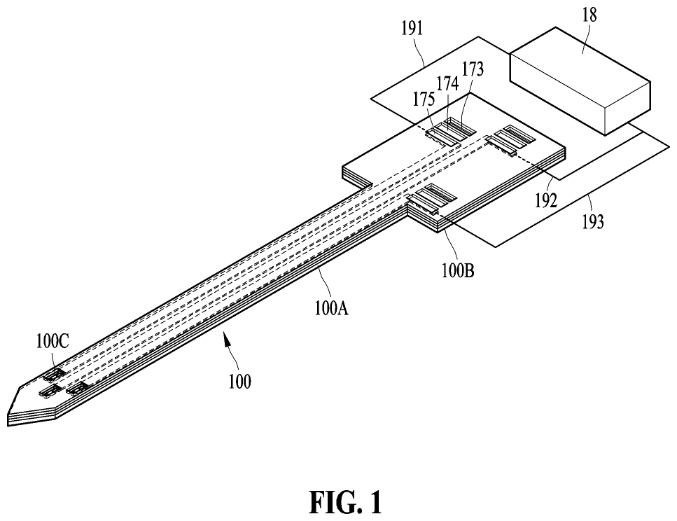

FIG. 1 illustrates a perspective view of an example probe apparatus for sensing an in vivo intracellular signal, according to one or more embodiments.

Although description of the examples and embodiments disclosed herein refers to in vivo sensing, extracellular sensing, and intracellular sensing, the examples and embodiments are not so limited. The examples and embodiments may be used for any kind of cell signal sensing. With this in mind, some examples and embodiments may be capable of both intracellular and extracellular signal sensing.

Referring to FIG. 1, a probe apparatus for sensing an in vivo intracellular signal 100 (hereinafter, referred to as the probe apparatus 100) is configured to be inserted into a subject and sense a signal of a cell of the subject; the sensed signal may be recorded. For example, the probe apparatus 100 may be inserted into a human brain. The probe apparatus 100 may both detect a location of a target cell through extracellular signal sensing and may sense, for recording, an intracellular signal of the target cell.

The probe apparatus 100 may adjust the physical location of the target cell by applying appropriate pressure through a flow channel. The probe apparatus 100 may include an extracellular electrode for the extracellular signal sensing and an intracellular electrode for the intracellular signal sensing. The probe apparatus 100 may include an apparatus head 100A having a relatively pointed shape, an apparatus body 100B having a relatively thick shape, and a cover main hole 100C formed penetrating the apparatus head 100A. The extracellular electrode and the intracellular electrode may be exposed to the outside through the cover main hole 100C. The target cell may penetrate/enter to the inside of the cover main hole 100C. The apparatus body 100B may include holes (e.g., a first terminal hole 173, a second terminal hole 174, and a channel hole 175). The dashed lines in FIG. 1 shows interior portions of pathways for electrical or fluid flow (as the case may be), and the solid lines show exterior portions of those pathways (as used herein, “fluid” includes both liquid and gaseous fluids).

A terminal of the extracellular electrode may be exposed to the outside through the first terminal hole 173. The terminal of the extracellular electrode may be electrically connected to an electronic device (not shown) provided externally. A terminal of the intracellular electrode may be exposed to the outside through the second terminal hole 174. The terminal of the intracellular electrode may also be electrically connected to the electronic device (not shown) provided externally. The probe apparatus 100 may include a flow channel. One end of the flow channel may be open toward the cover main hole 100C, and the other end of the flow channel may be open toward the channel hole 175, and the channel runs between the two end openings. There may be multiple sets of the first terminal hole 173, the second terminal hole 174, and the channel hole 175. The probe apparatus 100 may include a pump 18 and pump lines 191, 192, and 193. Each of the pump lines 191, 192, and 193 may be connected to a different channel hole. The pump 18 may apply different pneumatic pressure to the pump lines 191, 192, and 193. For example, at the same time (or in alternation) positive pressure may be applied to one of the plurality of pump lines 191, 192, and 193, and negative pressure may be applied to the other pump lines.

To summarize, the example probe apparatus 100 may have, among other things, multiple sensing sets that may share the structure of the same probe. A sensing set may include (i) a set of terminals (e.g., instances of first, second, and third terminal holes 173, 174, 175), (ii) a channel (e.g., two electrical channels and a fluid/flow channel), a hole/opening at the pointed tip of the probe, and, at the hole/opening, two electrodes for the respective electrical channels and an opening for the fluid channel. The pump 18 may provide fluid pressure to the fluid/flow channels. In the following description, although some elements are referred to in the singular, it is understood that, depending on context, description of the singular element may extend to other instances of the singular element (if they are present).

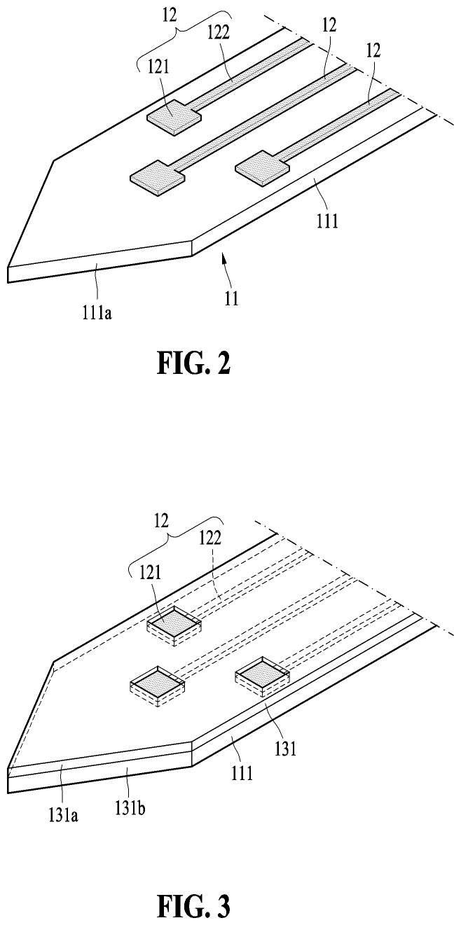

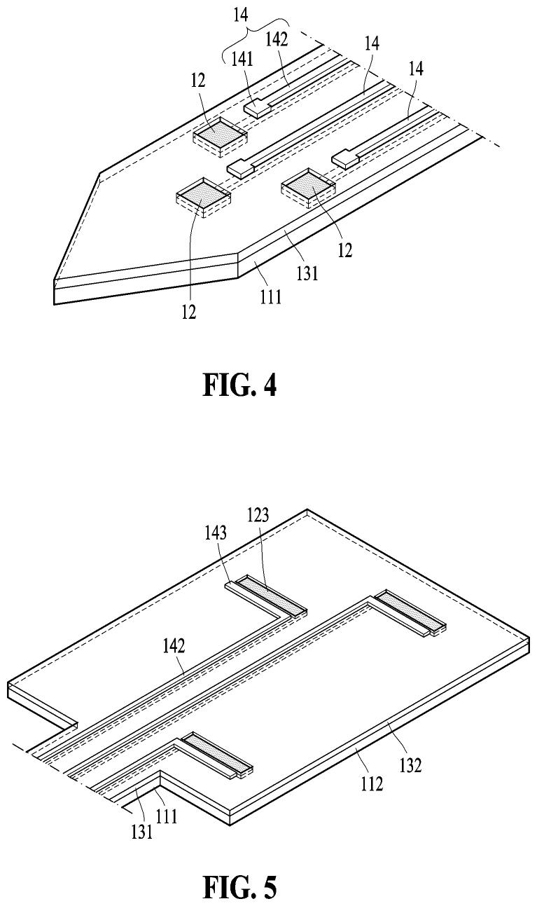

FIG. 2 illustrates a perspective view of an extracellular electrode disposed on a base layer, according to one or more embodiments. FIG. 3 illustrates an example of a first middle layer covering the extracellular electrode, according to one or more embodiments. FIG. 4 illustrates a perspective view of an example intracellular electrode disposed on the first middle layer, according to one or more embodiments. FIG. 5 illustrates a perspective view of the intracellular electrode disposed on the first middle layer, according to one or more embodiments.

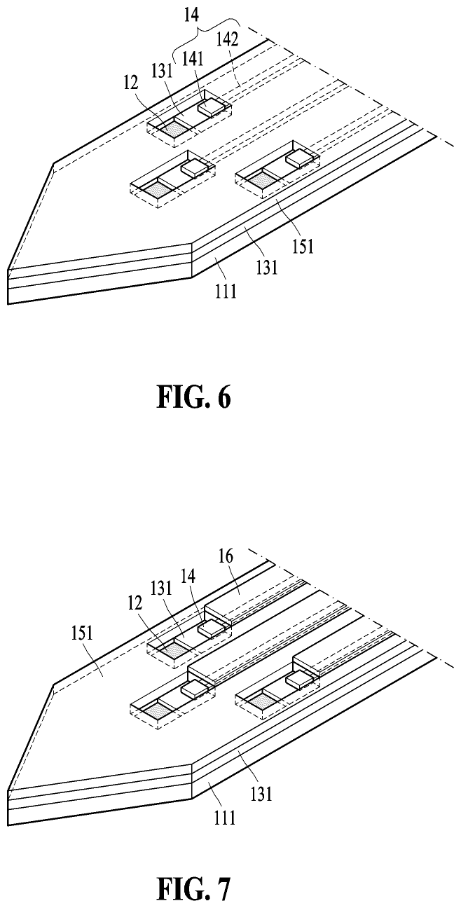

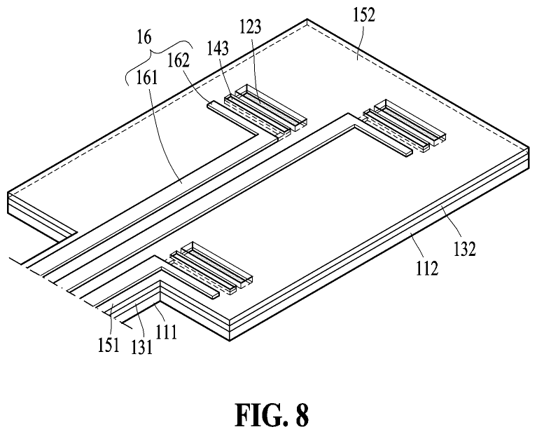

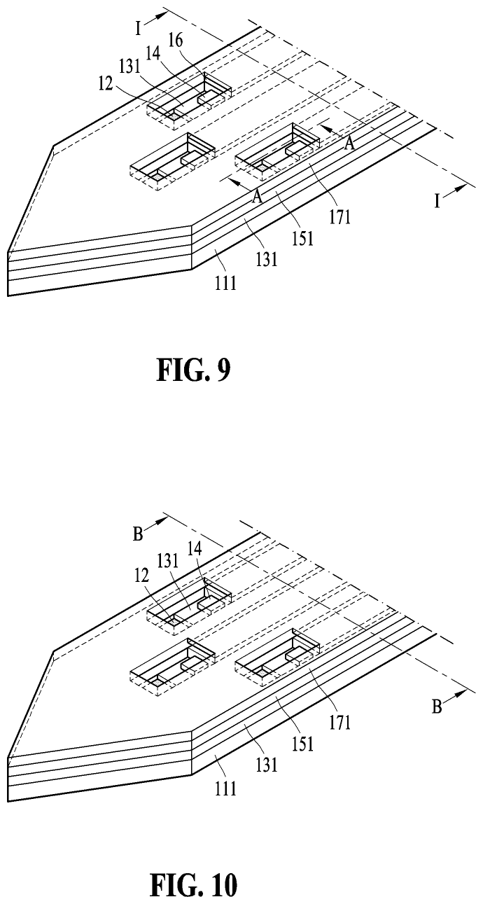

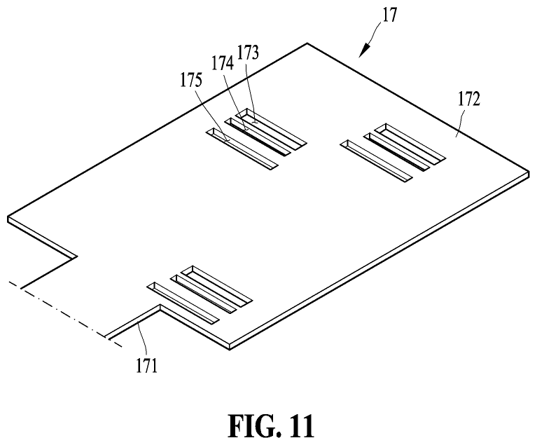

FIG. 6 illustrates a perspective view of a second middle layer covering the intracellular electrode, according to one or more embodiments. FIG. 7 illustrates of a perspective view of an example sacrificial layer disposed on the second middle layer, according to one or more embodiments. FIG. 8 illustrates a perspective view of the sacrificial layer disposed on the second middle layer, according to one or more embodiments. FIG. 9 illustrates a perspective view of a cover layer covering the sacrificial layer, according to one or more embodiments. FIG. 10 illustrates a perspective view after the sacrificial layer is dissolved and removed from FIG. 9, according to one or more embodiments. FIG. 11 illustrates a perspective view of the cover layer, according to one or more embodiments.

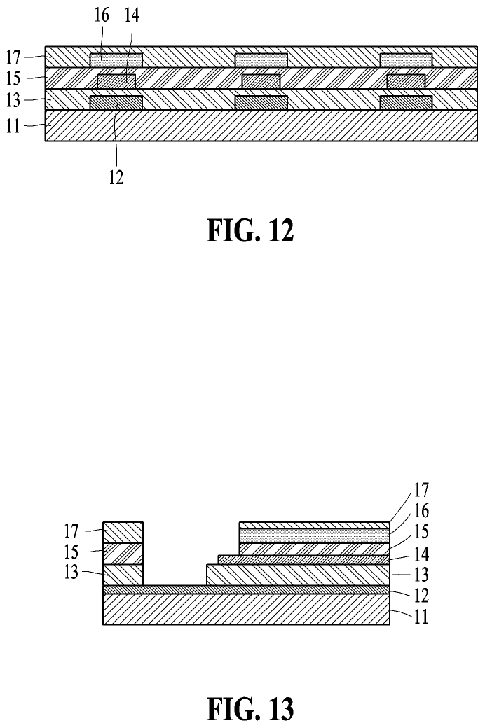

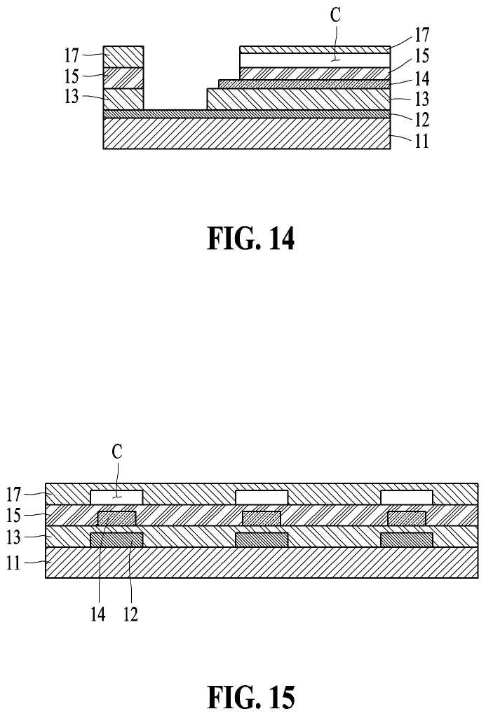

FIG. 12 illustrates a cross-sectional view taken along the line I-I of FIG. 9, according to one or more embodiments. FIG. 13 illustrates an example of a cross-sectional view taken along a line A-A of FIG. 9, according to one or more embodiments. FIG. 14 illustrates an example of a cross-sectional view taken along the line A-A of FIG. 9, in which a flow channel is formed, according to one or more embodiments. FIG. 15 illustrates an example of a cross-sectional view taken along a line B-B of FIG. 10, according to one or more embodiments.

Referring to FIGS. 2 to 15, a base layer 11 may include a base layer head 111 (e.g., insertable shaft and point) and a base layer body 112 (not necessarily insertable). A first electrical pathway may include an extracellular electrode 12 (which may include an extracellular electrode head 121 and an extracellular electrode line 122) and an extracellular electrode terminal 123.

The base layer head 111 may support the extracellular electrode head 121. The base layer body 112 may be linearly adjacent to the base layer head 111. The base layer body 112 may have a thicker shape than the base layer head 111 (e.g., width-wise as viewed from above). The base layer body 112 may support the extracellular electrode terminal 123. The extracellular electrode line 122 may be provided across the base layer head 111 and the base layer body 112. The base layer head 111 may include a base layer head tip 111a having a shape of which a width decreases toward an end portion (tapered/pointed). The base layer head tip 111a (and any other layers that have a head time portion) may be pointed by forming one or more bevels.

The extracellular electrode head 121 may have a thicker shape (e.g., a wider pad) than the extracellular electrode line 122. The extracellular electrode 12 may be disposed on the base layer 11. A longitudinal direction of the extracellular electrode terminal 123 may intersect with a longitudinal direction of the extracellular electrode line 122. For example, the extracellular electrode 12 may be a pad (extracellular electrode head 121) at the end of a line (extracellular electrode line 122).

A first middle layer 13 (see FIG. 12) may be disposed on the base layer 11 and may cover the extracellular electrode 12. The first middle layer and a first middle layer body 132 may be parts of the first middle layer 13 (may form a same plane). The first middle layer 13 may cover the extracellular electrode line 122 (the line sandwiched between the base and middle layers). The first middle layer 13 might not cover the extracellular electrode head 121 and the extracellular electrode terminal 123 (e.g., the first middle layer 13 may have a window the size and shape of the extracellular electrode head 121). The first middle layer 13 may include two holes through which to expose, to the outside, the extracellular electrode head 121 and the extracellular electrode terminal 123, respectively. The first middle layer head 131 may include a first middle layer head tip 131a having a shape of which a width decreases toward an end portion (tapered or pointed shape).

As noted above, a sensing set's channel may include a second electrical pathway. The second electrical pathway may include an intracellular electrode 14 (which may include an intracellular electrode head 141 and an intracellular electrode line 142) and an intracellular electrode terminal 143. The intracellular electrode head 141 may have a thicker shape than the intracellular electrode line 142. The intracellular electrode 14 may be disposed on the first middle layer 13. A longitudinal direction of the intracellular electrode terminal 143 may match a longitudinal direction of the intracellular electrode line 142. The intracellular electrode terminal 143 and the extracellular electrode terminal 123 may be provided spaced apart from each other and may be provided parallel to each other at adjacent locations.

A second middle layer 15 (see FIG. 12) may be disposed on the first middle layer 13 and may cover the intracellular electrode 14. The second middle layer 15 may cover the intracellular electrode line 142. The second middle layer 15 might not cover the intracellular electrode head 141 and the intracellular electrode terminal 143. The second middle layer 15 may include two holes through which to expose the intracellular electrode head 141 and the intracellular electrode terminal 143 to the outside. The hole exposing the intracellular electrode head 141 may be a window with the same size and shape as the intracellular electrode head 141 (which may be pad shaped). The second middle layer 15 may include the second middle layer head 151 and the second middle layer body 152.

A sacrificial layer 16 may include a sacrificial layer body 161 and a sacrificial layer base 162. The sacrificial layer body 161 may be disposed on the second middle layer 15. A longitudinal direction of the sacrificial layer body 161 may intersect/align with a longitudinal direction of the sacrificial layer base 162. The sacrificial layer base 162, the intracellular electrode terminal 143, and the extracellular electrode terminal 123 may be provided spaced apart from each other. The sacrificial layer base 162, the intracellular electrode terminal 143, and the extracellular electrode terminal 123 may be provided parallel to each other at adjacent locations. The sacrificial layer 16 may include, for example, a material that dissolves when in contact with a liquid. When the sacrificial layer 16 is dissolved and removed in a state in which the probe apparatus 100 (see FIG. 1) is completed, a space in which the sacrificial layer 16 was located may become a flow channel C.

A cover layer 17 may be disposed on the second middle layer 15 and may cover the sacrificial layer 16. The cover layer 17 may cover the sacrificial layer body 161. The cover layer 17 might not cover the sacrificial layer base 162. The cover layer 17 may include a hole through which to expose the sacrificial layer base 162 to the outside (allowing exposure to an etching liquid). The cover layer 17 may include a cover layer head 171, which may be a longitudinal member, a cover layer body 172 that is adjacent to the cover layer head 171 and has a shape of which a width is greater than a width of the cover layer head 171, the first terminal hole 173, the second terminal hole 174, the channel hole 175, and the cover main hole 100C (see FIG. 1).

The cover main hole 100C (see FIG. 1) may be formed penetrating the cover layer head 171 and may expose the extracellular electrode head 121 and the intracellular electrode head 141 to the outside.

The first terminal hole 173 may be formed penetrating the cover layer body 172 and may expose the extracellular electrode terminal 123 to the outside. The second terminal hole 174 may be formed penetrating the cover layer body 172 and may expose the intracellular electrode terminal 143 to the outside. The channel hole 175 may be formed penetrating the cover layer body 172 and may expose the flow channel C to the outside.

The hole formed in the first middle layer head 131, through which to expose the extracellular electrode 12 to the outside, may communicate (and align) with both the hole provided in the second middle layer head 151 and the hole provided in the cover layer head 171. The hole formed in the second middle layer head 151, through which to expose the intracellular electrode 14 to the outside, may communicate with the hole provided in the cover layer head 171.

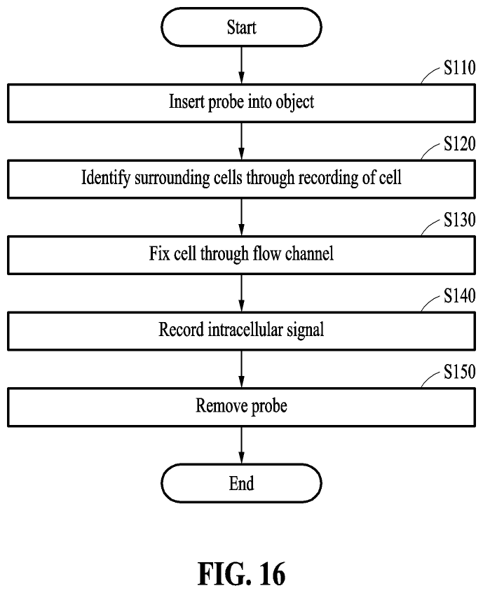

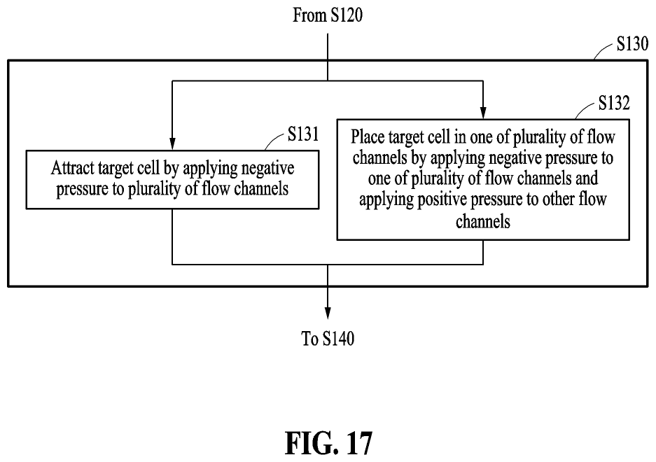

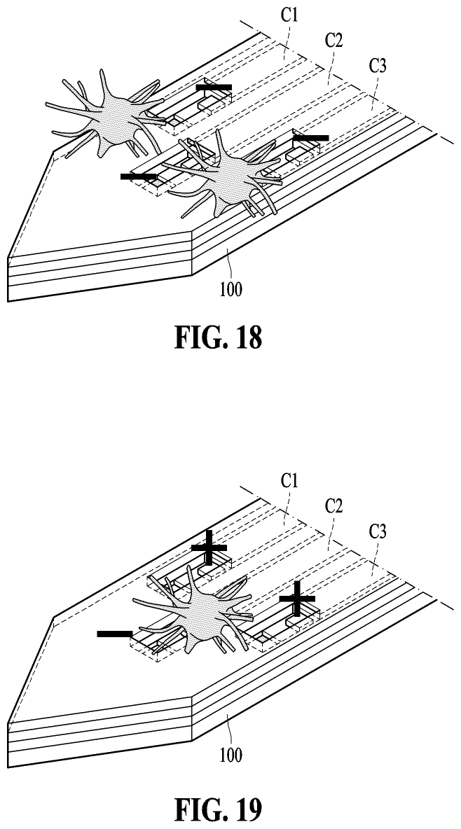

FIG. 16 illustrates an example of a method of using a probe apparatus for sensing an in vivo intracellular signal, according to one or more embodiments. FIG. 17 illustrates an example of an operation of fixing a cell through a flow channel, according to one or more embodiments. FIG. 18 illustrates an example of a target cell in the vicinity of the probe apparatus for sensing an in vivo intracellular signal, according to one or more embodiments. FIG. 19 shows the location of the target cell in the vicinity of the probe apparatus for sensing an in vivo intracellular signal, according to one or more embodiments.

Referring to FIGS. 16 to 19, a method of sensing an intracellular signal using the probe apparatus for sensing (for recording) an in vivo intracellular signal may include operation S110 of inserting a probe into an object, operation S120 of identifying surrounding cells through sensing of a cell, operation S130 of fixing a cell through a flow channel, operation S140 of sensing the intracellular signal, and operation S150 of removing the probe. Operations S110 and S150 may be performed by a user or a robot. Operation S120 may be performed with an extracellular electrode. Operation S130 may be performed with a pump and the flow channel (details of operation S130 are shown in FIG. 17). Operation S140 may be performed with an intracellular electrode.

Operation S130 may include two operations for first attracting a target cell and then placing the target cell. Specifically, operation S131 (see FIG. 17) may include attracting a target cell by applying negative pressure to each of the multiple flow channels (e.g., of multiple respective sensing sets) and operation S132 (see FIG. 17) may include placing/arranging the target cell in one flow channel of the plurality of flow channels by applying negative pressure to the one flow channel and applying positive pressure to the other flow channels. Operation S130 may include a variety of manners of operations other than operations S131 and S132.

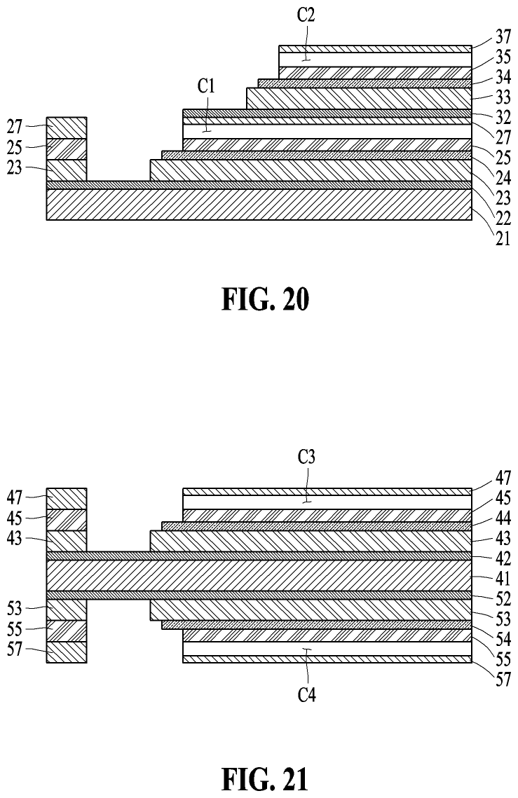

FIG. 20 illustrates a cross-sectional view of an example probe apparatus for sensing an in vivo intracellular signal, according to one or more embodiments.

Referring to FIG. 20, the probe apparatus for sensing an in vivo intracellular signal may include a first base layer 21, a first extracellular electrode 22, a first middle layer 23, a first intracellular electrode 24, a second middle layer 25, a first cover layer 27, a second extracellular electrode 32, a third middle layer 33, a second intracellular electrode 34, a fourth middle layer 35, and a second cover layer 37. The first base layer 21, the first extracellular electrode 22, the first middle layer 23, the first intracellular electrode 24, the second middle layer 25, the first cover layer 27, the second extracellular electrode 32, the third middle layer 33, the second intracellular electrode 34, the fourth middle layer 35, and the second cover layer 37 may be formed sequentially stacked. The probe apparatus for sensing an in vivo intracellular signal may include a first flow channel C1 and a second flow channel C2. The first flow channel C1 and the second flow channel C2 may be positioned in a same direction relative to the first base layer 21.

FIG. 21 illustrates a cross-sectional view of another example probe apparatus for sensing an in vivo intracellular signal, according to one or more embodiments.

Referring to FIG. 21, the probe apparatus for sensing an in vivo intracellular signal may include a base layer 41, a first extracellular electrode 42, a first middle layer 43, a first intracellular electrode 44, a second middle layer 45, a first cover layer 47, a second extracellular electrode 52, a third middle layer 53, a second intracellular electrode 54, a fourth middle layer 55, and a second cover layer 57. The base layer 41, the first extracellular electrode 42, the first middle layer 43, the first intracellular electrode 44, the second middle layer 45, and the first cover layer 47 may be formed sequentially stacked. The second extracellular electrode 52, the third middle layer 53, the second intracellular electrode 54, the fourth middle layer 55, and the second cover layer 57 may be formed sequentially stacked. The probe apparatus for sensing an in vivo intracellular signal may include a third flow channel C3 and a fourth flow channel C4. The third flow channel C3 and the fourth flow channel C4 may be positioned in opposite directions relative to the base layer 41.

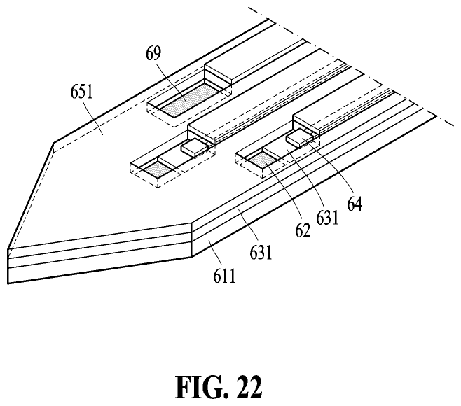

FIG. 22 illustrates a perspective view of an example probe apparatus for sensing an in vivo intracellular signal, according to one or more embodiments.

Referring to FIG. 22, a first middle layer head 631 may be stacked on a base layer head 611. A second middle layer head 651 may be stacked on the first middle layer head 631. An extracellular electrode 62 and an intracellular electrode 64 may be paired, and there may be two such pairs. In a position (or sensing set) where the extracellular electrode 62 and the intracellular electrode 64 are not provided, an additional electrode 69 may be provided. The additional electrode 69 may function as a reference electrode or a stimulation electrode. For example, there may be multiple additional electrodes 69. For example, the additional electrode 69 may be provided in a plural number (e.g., two), and one of the two additional electrodes may be a reference electrode and the other may be a stimulation electrode. It should be noted that the number of the extracellular electrode 62, the intracellular electrode 64, and the additional electrode 69 is not limited. Likewise, it should be noted that the number of flow channels is not limited.

A probe apparatus for sensing an in vivo intracellular signal includes a base layer, an extracellular electrode disposed on the base layer and comprising an extracellular electrode head, an extracellular electrode line formed extending from the extracellular electrode head, and an extracellular electrode terminal formed extending from the extracellular electrode line, a first middle layer that is disposed on the base layer and configured to cover the extracellular electrode line, an intracellular electrode which is disposed on the first middle layer, which is provided spaced apart from the extracellular electrode, and which comprises an intracellular electrode head, an intracellular electrode line formed extending from the intracellular electrode head, and an intracellular electrode terminal formed extending from the intracellular electrode line, a second middle layer that is disposed on the first middle layer and configured to cover the intracellular electrode line, a cover layer disposed on the second middle layer, and a flow channel provided between the second middle layer and the cover layer.

The cover layer may include a cover layer head, which may be a longitudinal member, and a cover layer body that is adjacent to the cover layer head and has a shape of which a width is greater than a width of the cover layer head.

The cover layer may further include a cover main hole that is formed penetrating the cover layer head and exposes the extracellular electrode head and the intracellular electrode head each to the outside.

The cover layer may further include a first terminal hole that is formed penetrating the cover layer body and exposes the extracellular electrode terminal to the outside.

The cover layer may further include a second terminal hole that is formed penetrating the cover layer body, exposes the intracellular electrode terminal to the outside, and is provided at a location spaced apart from the first terminal hole.

The cover layer may further include a channel hole that is formed penetrating the cover layer body and communicates the flow channel to the outside.

The flow channel may be provided with one end open toward the cover main hole and another end open toward the channel hole.

The base layer may include a base layer head that is configured to support the extracellular electrode head and that overlaps the cover layer head and may include a base layer body that is adjacent to the base layer head and configured to support the extracellular electrode terminal.

The first middle layer may include a first middle layer head which has a hole that communicates with the cover main hole, which is configured to support the intracellular electrode head, and which overlaps the cover layer head and may include a first middle layer body that is adjacent to the first middle layer head and configured to support the intracellular electrode terminal.

The second middle layer may include a second middle layer head which has a hole that communicates with the cover main hole and which overlaps the cover layer head and may include a second middle layer body adjacent to the second middle layer head.

There may be multiple pairs of he extracellular electrode and the intracellular electrode.

The extracellular electrodes may have different lengths.

A longitudinal direction of the extracellular electrode line and a longitudinal direction of the intracellular electrode terminal may be provided to intersect with each other.

Based on a stacking direction of the base layer (e.g., a normal thereof), the first middle layer, the second middle layer, and the cover layer, a width of the flow channel may be formed to be less than a width of a portion of the second middle layer that does not overlap the flow channel.

The probe apparatus may further include a pump configured to generate pneumatic pressure and a pump line connecting the pump to the flow channel.

A probe apparatus for sensing an in vivo intracellular signal includes a base layer, an intracellular electrode disposed on the base layer, a first middle layer that is disposed on the base layer and configured to cover at least a portion of the intracellular electrode, an extracellular electrode disposed on the first middle layer and provided spaced apart from the intracellular electrode, a second middle layer that is disposed on the first middle layer and configured to cover at least a portion of the extracellular electrode, a sacrificial layer disposed on the second middle layer and provided spaced apart from the intracellular electrode and the extracellular electrode, the sacrificial layer comprising a material that dissolves in a liquid (e.g., an etching liquid), and a cover layer disposed on the sacrificial layer.

Based on a stacking direction of the base layer, the first middle layer, the second middle layer, and the cover layer, a width of the sacrificial layer may be formed to be less than the width of the portion of the second middle layer that does not overlap the sacrificial layer. In another example, the width of the sacrificial layer may be greater than the width of the portion of the second middle layer that does not overlap the sacrificial layer.

The cover layer may include a cover main hole that exposes, to the outside, each of an end portion of the extracellular electrode and an end portion of the intracellular electrode.

The cover layer may further include a first terminal hole that exposes another end portion of the extracellular electrode to the outside and a second terminal hole that exposes another end portion of the intracellular electrode to the outside and is provided at a location spaced apart from the first terminal hole.

A method of sensing an intracellular signal using a probe apparatus for sensing an in vivo intracellular signal, wherein the probe apparatus includes a base layer, an extracellular electrode disposed on the base layer, a first middle layer that is disposed on the base layer and configured to cover the extracellular electrode, an intracellular electrode that is disposed on the first middle layer, a second middle layer that is disposed on the first middle layer and configured to cover the intracellular electrode, a cover layer disposed on the second middle layer, and a flow channel provided between the second middle layer and the cover layer, includes performing, by the extracellular electrode, extracellular signal sensing of a target cell, fixing, by the flow channel, the target cell, and performing, by the intracellular electrode, intracellular signal sensing of the target cell.

While the examples are described with reference to a limited number of drawings, it will be apparent to one of ordinary skill in the art that various alterations and modifications in form and details may be made in these examples without departing from the spirit and scope of the claims and their equivalents. For example, suitable results may be achieved if the described techniques are performed in a different order and/or if components in a described system, architecture, device, or circuit are combined in a different manner and/or replaced or supplemented by other components or their equivalents.

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents.

Therefore, in addition to the above disclosure, the scope of the disclosure may also be defined by the claims and their equivalents, and all variations within the scope of the claims and their equivalents are to be construed as being included in the disclosure.

What is claimed is:

1. A probe apparatus for sensing cellular signals, the apparatus comprising:
a base layer;
a first electrode disposed on the base layer and comprising a first electrode head, a first electrode line extending from the first electrode head, and a first electrode terminal extending from the extracellular electrode line;
a first middle layer that is disposed on the base layer and is configured to cover the first electrode line;
a second electrode that is disposed on the first middle layer and provided spaced apart from the first electrode, wherein the second electrode comprises a second electrode head, a second electrode line formed extending from the second electrode head, and a second electrode terminal extending from the second electrode line;
a second middle layer that is disposed on the first middle layer and is configured to cover the second electrode line;
a cover layer disposed on the second middle layer; and
a fluid-flow channel provided between the second middle layer and the cover layer.

2. The apparatus of claim 1, wherein the cover layer comprises:
a cover layer head, which is a longitudinal member; and
a cover layer body that is adjacent to the cover layer head and has a width greater than a corresponding width of the cover layer head.

3. The apparatus of claim 2, wherein the cover layer further comprises:
a cover main hole that is formed penetrating the cover layer head and exposes the first electrode head and the second electrode head each to the outside.

4. The apparatus of claim 2, wherein the cover layer further comprises:
a first terminal hole that is formed penetrating the cover layer body and that exposes the first electrode terminal to the outside.

5. The apparatus of claim 4, wherein the cover layer further comprises:
a second terminal hole that is formed penetrating the cover layer body, that exposes the second electrode terminal to the outside, and that is provided at a location spaced apart from the first terminal hole.

6. The apparatus of claim 3, wherein the cover layer further comprises:
a channel hole that is formed penetrating the cover layer body and communicates the fluid-flow channel to the outside.

7. The apparatus of claim 6, wherein the flow channel is provided with one end open toward the cover main hole and another end open toward the channel hole.

8. The apparatus of claim 3, wherein the base layer comprises:
a base layer head that is configured to support the first electrode head and overlaps the cover layer head; and
a base layer body that is connected to the base layer head and is configured to support the first electrode terminal.

9. The apparatus of claim 3, wherein the first middle layer comprises:
a first middle layer head that has a hole that communicates with the cover main hole, that is configured to support the second electrode head, and that overlaps the cover layer head; and
a first middle layer body that is connected to the first middle layer head and is configured to support the second electrode terminal.

10. The apparatus of claim 3, wherein the second middle layer comprises:
a second middle layer head that has a hole that communicates to the cover main hole and that overlaps the cover layer head; and
a second middle layer body adjacent to the second middle layer head.

11. The apparatus of claim 1, wherein there are multiple first electrodes and multiple second electrodes.

12. The apparatus of claim 11, wherein the first electrodes have different lengths with respect to each other.

13. The apparatus of claim 1, wherein a longitudinal direction of the first electrode line and a longitudinal direction of the second electrode terminal are provided to intersect with each other.

14. The apparatus of claim 1, wherein a width of the flow channel is formed to be less than a width of a portion of the second middle layer that does not overlap the flow channel.

15. The apparatus of claim 1, further comprising:
a pump configured to generate pneumatic pressure; and
a pump line connecting the pump with the flow channel.

16. A probe apparatus for sensing a cellular signal, the apparatus comprising:
a base layer;
a second electrode disposed on the base layer;
a first middle layer that is disposed on the base layer and configured to cover at least a portion of the second electrode;
a first electrode disposed on the first middle layer and provided spaced apart from the second electrode;
a second middle layer that is disposed on the first middle layer and configured to cover at least a portion of the first electrode;
a sacrificial layer disposed on the second middle layer and provided spaced apart from the second electrode and the first electrode, the sacrificial layer comprising a material that dissolves in a liquid; and
a cover layer disposed on the sacrificial layer.

17. The apparatus of claim 16, wherein a width of the sacrificial layer is formed to be less than a width of a portion of the second middle layer that does not overlap the sacrificial layer.

18. A cell probe comprising:
a pointed shaft and a body together comprising a first sensing set;
the pointed shaft including a first opening at a pointed end of the shaft;
the first sensing set comprising a first fluid pathway, a first electrical pathway, and a second electrical pathway;
the first fluid pathway comprising a first tunnel passing longitudinally through the shaft and having one end opening at the first hole;
the first electrical pathway comprising a first electrode pad exposed via the first hole and a first electrical line connected to the first electrode pad; and
the second electrical pathway comprising a second electrode pad exposed via the first hole and a second electrical line connected to the second electrode pad.

19. The cell probe of claim 18, wherein
the first electrical line is connected to a first electrical terminal comprised in the body;
the second electrical line is connected to a second electrical terminal comprised in the body; and
wherein the cell probe further comprises a pump connected with the first tunnel to allow fluid flow between the pump and the one end of the first tunnel.

20. The cell probe of claim 19, wherein the pointed shaft includes a second opening at the pointed end of the shaft, wherein the pointed shaft and the body together comprise a second sensing set, and wherein the second sensing set comprises:
a second fluid pathway, a third electrical pathway, and a fourth electrical pathway;
the second fluid pathway comprising a second tunnel passing longitudinally through shaft and having one end opening at the second hole;
the third electrical pathway comprising a third electrode pad exposed via the second hole and a third electrical line connected to the third electrode pad; and
the fourth electrical pathway comprising a fourth electrode pad exposed via the second hole and a fourth electrical line connected to the fourth electrode pad.
